(19) United States
(12) Patent Application Publication
(10) Pub. No.: US 2021/0195870 A1
(43) Pub. Date: Jul. 1, 2021

(54) A CONSUMABLE CARTRIDGE REMOVABLY ATTACHABLE TO AN ANIMAL MARKING APPARATUS AND A METHOD FOR MARKING AN ANIMAL

(71) Applicant: Somark Group Ltd, SYDNEY, New South Wales (AU)

(72) Inventors: Adrian Knight, SYDNEY, New South Wales (AU); Paul Donohoe, SYDNEY, New South Wales (AU); Michael Potas, SYDNEY, New South Wales (AU); Lee Blattman, Sydney, New South Wales (AU); Robert Seaman, Sydney, New South Wales (AU); David Jones, Sydney, New South Wales (AU)

(73) Assignee: Somark Group Ltd, SYDNEY, New South Wales (AU)

(21) Appl. No.: 16/755,316

(22) PCT Filed: Oct. 12, 2018
(86) PCT No.: PCT/AU2018/051106

§ 371 Date: Apr. 10, 2020

Related U.S. Application Data
(60) Provisional application No. 62/571,750, filed on Oct. 12, 2017.

(51) Int. Cl. A01K 11/00; A61M 37/00

(52) U.S. Cl. CPC A01K 11/005; A61M 37/0084; A61M 2250/00; A61M 2039/2433; A61M 2205/52; A61M 2205/60; A61M 2205/123

(57) ABSTRACT

Disclosed herein is a consumable cartridge removably attachable to an animal marking apparatus. The consumable cartridge comprises a movably mounted needle and a biasing element operatively coupled to the movably mounted needle to withdraw the needle to a withdrawn position. Also disclosed herein is a method for marking an animal.

### TECHNICAL FIELD

The disclosure herein generally relates to a consumable cartridge removably attachable to an animal marking apparatus and a method for marking an animal.

### BACKGROUND

Animals including laboratory animals generally need to be identified. An animal may be tattooed with identity information in the form of a plurality of symbols, have an identity tag attached, or have an ear notched. The identity of animals may be determined by inspecting the ear identification notches, attached tag, or tattoo. This may be time consuming and/or difficult in some circumstances, for example when tattooing hundreds or thousands of mice in a facility. These operations may require skilled technicians. Mistakes may occur, and accuracy may be variable or impossible to achieve.

A machine may receive a part of the animal and tattoo predetermined identity information on the part. Tattoing the animal may consume needles in the form of tattooing needles and pigment in the form of tattooing ink. The needles and the pigment can be a significant cost, can be fiddly, messy and time consuming to replace. Care must be taken not to transfer pathogens or diseases.

The tattoo needle can stick into a user, which may be a health and safety issue, particularly when physiologically active substances, for example pathogens, have been administered to the animal.

The tattoo needle can blunt. A user attempt to use a tattoo needle after it has blunted may damage and/or stress the animal.

In the context of this specification, an animal may be generally any suitable non-human animal or human, including a laboratory animal, a rodent, a rat, a mouse, a cat, a dog, a rabbit, a bird including a poultry bird, livestock including cattle, sheep and horses, a zoo animal or generally any type of animal.

### SUMMARY

Disclosed herein is a consumable cartridge removably attachable to an animal marking apparatus. The consumable cartridge comprises a movably mounted needle and a biasing element operatively coupled to the movably mounted needle to withdraw the needle. The comprisable cartridge comprises a needle actuator arranged to be moved by the animal marking apparatus during attachment of the cartridge and when so moved the needle actuator moves the movably mounted needle outwards. The consumable cartridge comprises a pigment reservoir comprising a tattoo pigment, the pigment reservoir being in fluid communication with a pigment nozzle.

An embodiment comprises a needle housing defining a cavity in which the movable mounted needle is withdrawn into and extending between an open end from which the needle actuator protrudes and another open end for passage of the needle. The needle actuator may be arranged to be inwardly moved by the animal marking apparatus and so move the tip of the needle out of the other open end.

In an embodiment, the biasing member comprises a compression spring. The spring may be coiled around the needle. The spring may have an end seated adjacent the other open end.

In an embodiment, the spring comprises another end that outwardly pushes the needle actuator.

In an embodiment, the needle actuator comprises an end cap attached to the movably mounted needle and having a seat for the other end of the spring.

An embodiment comprises a cartridge joint member configured to be rotated into engagement with an animal marking apparatus joint member to form a connecting joint between the consumable cartridge and the animal marking apparatus. The needle actuator may be arranged to be inwardly moved by a cam surface of the animal marking apparatus when the cartridge joint member is so rotated into engagement.

In an embodiment, the connecting joint comprises at least one of a connecting bayonet joint, and a connecting screw joint.

In an embodiment, the pigment reservoir comprises a plunger for moving the pigment.

In an embodiment, the plunger is externally actuatable by the animal marking apparatus.

In an embodiment, the pigment nozzle is adjacent to a tip of the needle.

In an embodiment, the pigment nozzle is directed at at least one of the movably mounted needle subsequent to being outwardly moved, and when removably attached to the animal marking apparatus, a point within the animal marking apparatus at which the animal present.

In an embodiment, in the pigment nozzle comprises a duck-bill valve. Alternatively or additionally, the pigment nozzle comprises a hollow needle.

In an embodiment, the pigment nozzle is dimensioned to be sealed by pigment surface tension.

An embodiment comprises a mixing element disposed in the pigment reservoir.

An embodiment comprises a mobile mixing element disposed in the pigment reservoir.

An embodiment comprises non-volatile memory and electrical contacts in electrical communication with the non-volatile memory for the animal marking machine to at least one of read and write to the non volatile memory. The non-volatile memory may comprise at least one of consumable cartridge identification information, information indicative of the number of pigment applications, pigment colour information, needle type identification information, date information, number of symbols marked on the animal, and total operation time information.

In an embodiment, the pigment reservoir holds no ejectable pigment when the needle is blunted through use.

Disclosed herein is a method for marking an animal. The method comprising the step of removably attaching a consumable cartridge to an animal marking apparatus during which a needle actuator of the consumable cartridge is moved by the animal marking apparatus to outwardly move a movably mounted needle that is inwardly biased. The method comprising the step of ejecting a pigment from a pigment reservoir of the consumable cartridge. The method comprising the step of and penetrating an animal received by the animal marking apparatus with the movable mounted needle for ingress of the ejected pigment into the animal.

In an embodiment, the animal is a non-human animal.

In an embodiment, the animal is a human.

Any of the various features of each of the above disclosures, and of the various features of the embodiments described below, can be combined as suitable and desired.

### BRIEF DESCRIPTION OF THE FIGURES

Embodiments will now be described by way of example only with reference to the accompanying figures in which:

FIG. 1 shows a perspective view of an embodiment of a consumable cartridge removably attachable to an animal marking apparatus.

FIG. 1a shows a cut-away view of the consumable cartridge of FIG. 1.

FIG. 2 shows a longitudinal section view of the consumable cartridge of FIG. 1.

FIG. 3 shows a perspective view of an example of the animal marking apparatus.

FIG. 4 shows an example of an animal for marking by the animal marking apparatus of FIG. 3.

FIG. 5 shows a side elevational view of an animal restraint for restraining the animal of FIG. 4 and subsequent insertion into the animal apparatus of FIG. 3.

FIG. 6 shows an example of a contamination barrier sleeve used with the apparatus of FIG. 3.

FIGS. 7 and 8 show steps of an embodiment of a method for removably attaching the consumable cartridge to the animal marking apparatus of FIG. 3.

FIG. 9 shows a perspective view corresponding to FIG. 8.

FIG. 10 shows a flow chart of an embodiment of a method for marking an animal.

FIG. 11 shows a perspective view of the needle of the consumable cartridge in a parked position.

FIG. 12 shows a section view of another embodiment of a consumable cartridge removably attachable to an animal marking apparatus.

FIGS. 13 and 14 show different perspective views of the consumable cartridge of FIG. 12.

### DESCRIPTION OF EMBODIMENTS

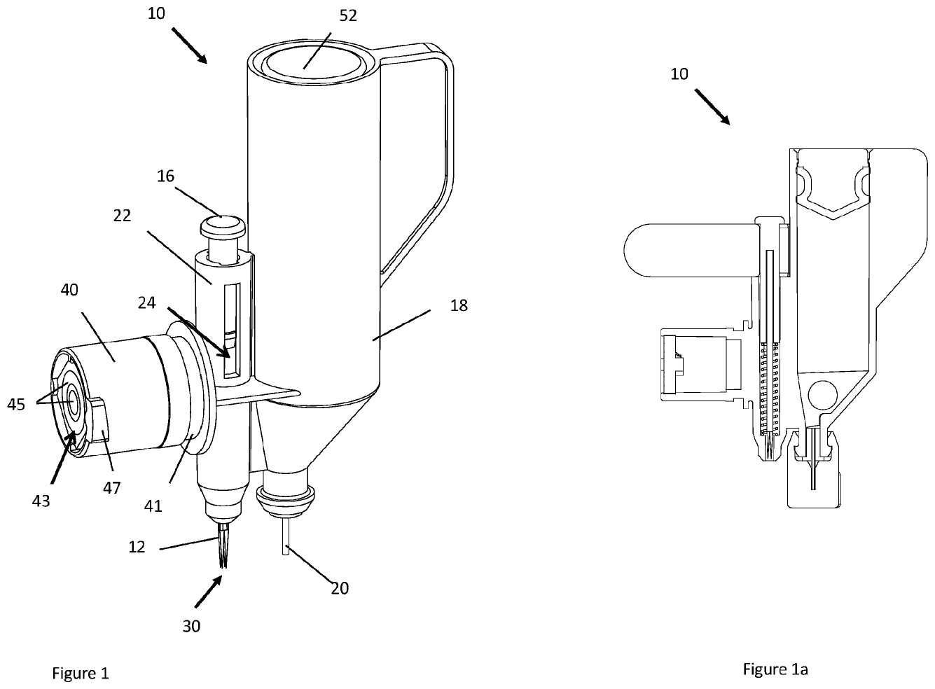

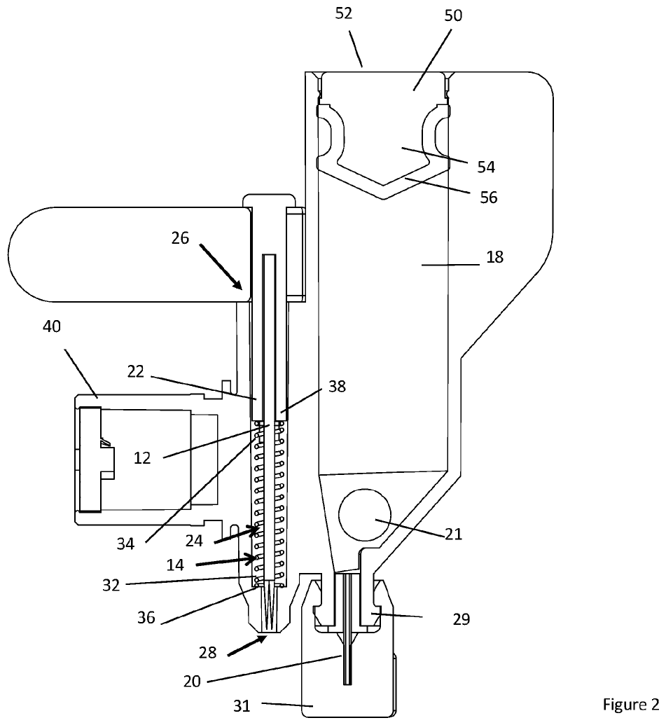

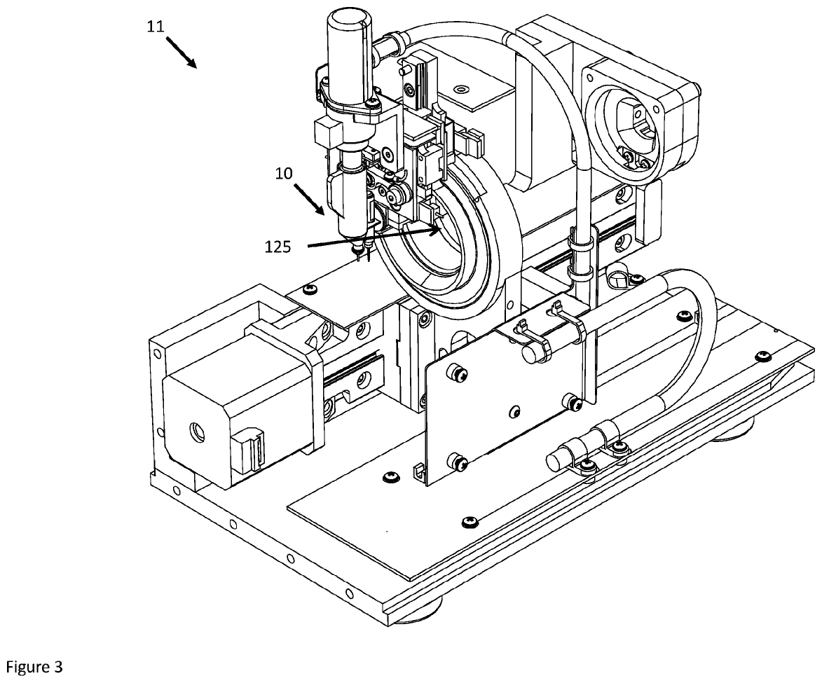

FIGS. 1 and 2 show respectively a perspective view and a longitudinal section view of a consumable cartridge removably attachable to an animal marking apparatus, the consumable cartridge being indicated by the numeral 10. A perspective view of an example of an animal marking apparatus 11 to which the consumable cartridge 10 is removably attached in shown in FIG. 3. The consumable cartridge 10 comprises a movably mounted needle 12 and a biasing element 14 operatively coupled to the movably mounted needle 12 to withdraw the needle to a withdrawn position. The needle is withdrawn in FIG. 2 and extended in FIG. 1. The consumable cartridge 10 comprises a needle actuator 16, that in this but not all embodiments is configured to hold the needle 12, arranged to be moved by the animal marking apparatus 11 during attachment of the cartridge. When the needle actuator 16 is so moved by the animal marking apparatus 11, the movably mounted needle 12 is moved outwardly by the needle actuator 16 to an extended position. The consumable cartridge 10 comprises a pigment reservoir 18 comprising tattoo pigment. The pigment reservoir 18 is in fluid communication with a pigment nozzle 20, which may be in the form of a needle.

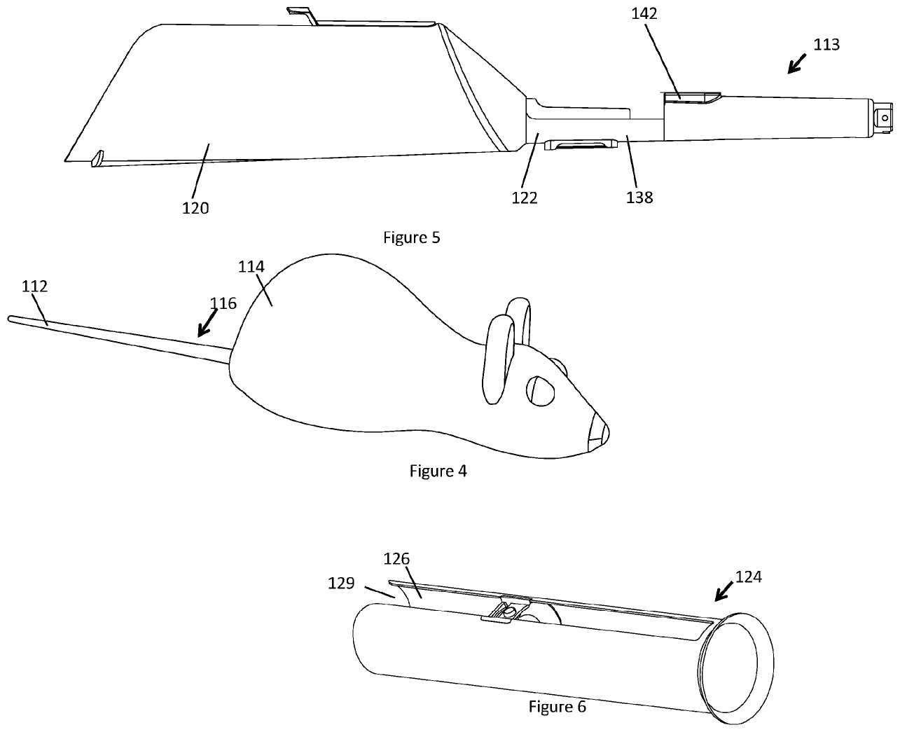

The apparatus 11 if configured to mark a part 112 of a rodent 114 in the form of a mouse or rat, shown in FIG. 4, however the animal may be any suitable animal, for example a bird, dog or cattle, and the part 112 may be any suitable part of the animal 10, for example foot, ear or appendage. In the present embodiment, the part 112 is the tail of the rodent 114. FIG. 5 shows an example of an animal restraint 118 comprising a restraint portion 120 configured to restrain the rodent 114, and a tail presentation portion 122 that is elongated for supporting the length of the tail 112 and configured to present the surface 116 of the tail 112 for penetration by the needle 12. In this but not all embodiments, the surface 116 is a dorsal surface of the tail 112. FIG. 6 shows a perspective view of an example of a restraint receiver 124. The animal restraint 118 is received by the restraint receiver 124 of the apparatus 11, the restraint receiver 124 comprises a sleeve in the form of a splatter proof sleeve shown in FIG. 6 and in use is received within an annular opening 125 of the system 10. The sleeve 124 comprises a tube 126 have a slot 129 along its side wall. In use, the slot 129 provides a passage for the needle 12, providing access to the part 112 and surface 116. The splatter proof sleeve 124 is a barrier to splatter from the penetration, which may result in cross-contamination of mice processed by the system 10, or degrade or damage the system 10. The splatter proof sleeve may comprise stainless steel, a polymer, or generally any suitable material.

The consumable cartridge 10 has a needle housing 22 defining a cavity 24 in which the movably mounted needle 12 is withdrawn into and extending between an open end 26 from which the needle actuator protrudes and another open end 28 for passage of the needle 12. The needle actuator 16 is arranged to be inwardly moved by the animal marking apparatus 11 and so move the tip 30 of the needle 12 out of the other open end 28 when the consumable cartridge 10 is removably attached to the animal marking apparatus 11.

The biasing member 14 comprises a compression spring that is coiled around the needle 12 and having an end 32 seated adjacent the other open end 28. The end 32 of the spring is seated in a seat 36 in the form of an internal rebate formed in the needle housing 22. The spring 14 comprises another end 34 that outwardly pushes the needle actuator 16. The needle actuator 16 comprises an end cap attached to the movably mounted needle 12 and having a seat 38 in the form of an interior end face for the other end 34 of the spring 14. In another embodiment, the spring is not coiled around the needle, but compressed between the needle 12 and the needle actuator 16. The biasing member 14 may comprise silicone, and may be in the form of a silicone spring.

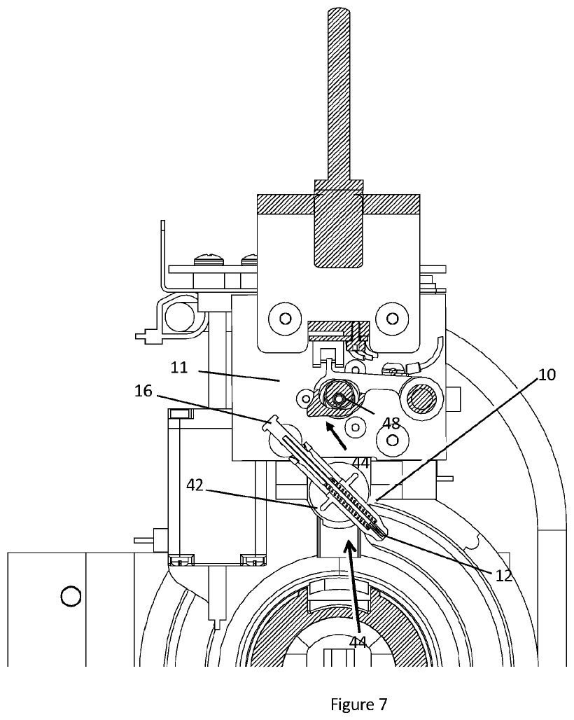

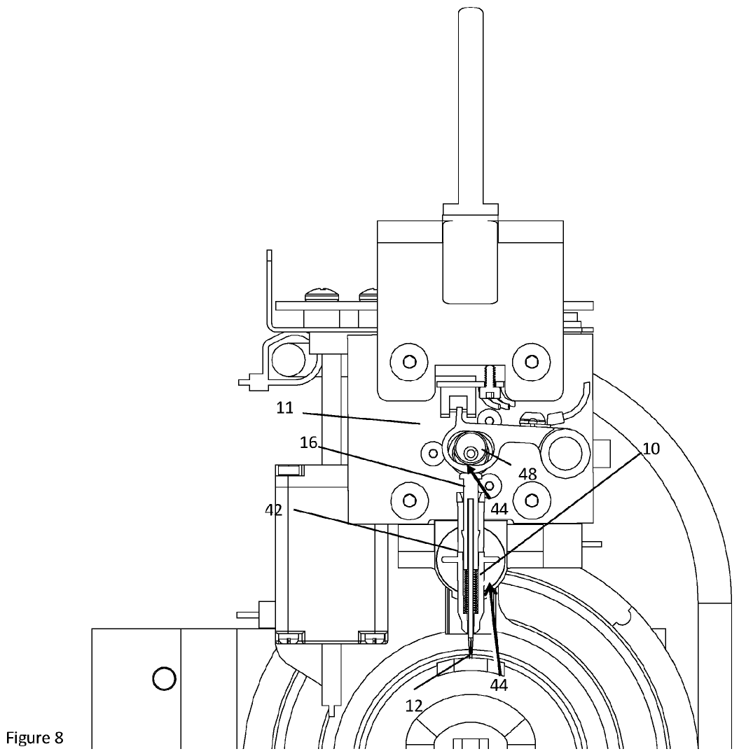

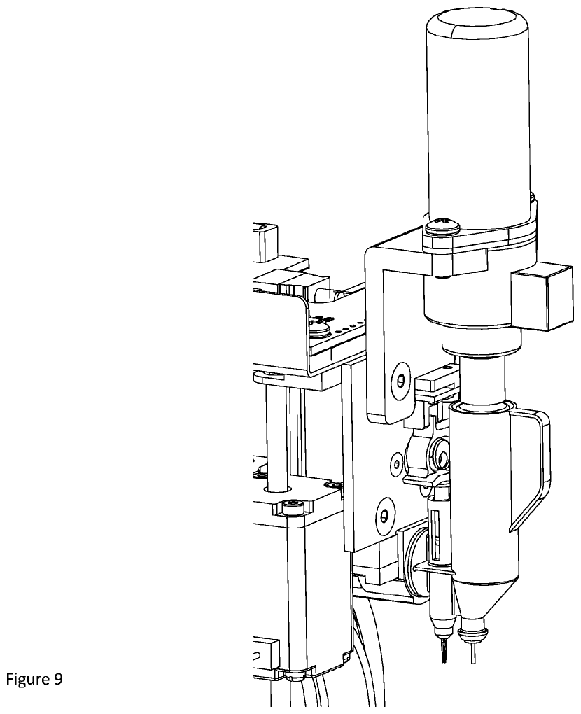

The cartridge has a cartridge joint member 40 configured to be rotated into engagement with an animal marking apparatus joint member to form a connecting joint in the form of a connecting bayonet joint between the consumable cartridge 10 and the animal marking apparatus 11. The cartridge joint member 40 comprises bayonet pins 47. The joint member 40 has circumferential seat 41 for a seal in the form of an O-ring seal. FIGS. 7 and 8 show steps of an embodiment of a method for removably attaching the consumable cartridge 11 to the animal marking apparatus of FIG. 3. As shown in FIG. 7, in a step the cartridge joint member 40 comprising a plug is inserted into the animal apparatus joint member 42 comprising a socket. The consumable cartridge 10 is rotated relative to the animal marking apparatus 11 to lockingly engage the cartridge joint member 40 and the animal apparatus joint member 42 to form a joint 44 in the form of a bayonet joint. The socket 42 and the plug 40 may be interchanged. It will be appreciated that generally any suitable rotary joint may be used, for example but not limited to a connecting screw joint. The protruding needle actuator 16 is for engagement with a cam surface 46 of the animal marking apparatus 11 when so rotated into engagement. As the consumable cartridge 10 is rotated to lockingly engage the cartridge joint member 40 and the animal apparatus joint member 42, the needle actuator 16 engages the cam surface 46 and moves inward to follow the cam surface 44. The inwardly moving needle actuator 16 moves the needle 12 so that it is extended. The cam surface 44 is also for transmitting an oscillatory motion to the needle actuator 16 for repeated puncturing of a surface of the animal. Pigment enters the animal via the punctures so formed. The cam surface 44 is an outer surface of a collar 46 which engages an eccentric 48 within the collar 46 and coupled to an electric motor.

The pigment reservoir 18 comprises a plunger 50 for ejecting the pigment. The plunger 50 comprises a head 54 in the form of a conical head having a seal 56, and an exposed engagable surface 52 at the other end for actuation by the animal marking apparatus 11. The plunger is actuated by a stepper motor to eject a volume of pigment in the range of 2 μL and 10 μL, however more or less pigment may be ejected in alternative embodiments. The pigment is in the form of a liquid having suspended pigment particles, however it may comprise a pigment powder, a pigment solution, or generally any suitable form of pigment. The pigment may separate when still. The pigment nozzle 20 is adjacent the tip 30 of the needle. The pigment nozzle 20 is orientated to deposit pigment on the surface 116 via the needle 12 when extended. The pigment may run down the needle 12 to the tip 30 thereof, and the pigment may drop off the tip 30. Alternatively, the needle may be towered so that the drop of pigment contacts the surface 116, and the drop of pigment drawn away from the tip 30 by surface tension. Alternatively, the pigment nozzle 20 may be directed at a point within the animal marking apparatus at which the animal when received is present, for depositing the pigment directly onto the surface 116. A line of pigment may be drawn on the surface. The cartridge may be translated along the tail as pigment is ejected to draw the line of pigment. The tip of the needle is square to the needle shaft, unlike a hypodermic needle that has a tip that is oblique to the needle shaft. Consequently, the droplet formed is inline with the needle and not to a side of the needle. This may improve drop formation and/or pigment deposition. As the needle 12 penetrates the animal 114, the pigment on the needle 12 and/or surface 116 is transferred into the animal, and captured therein when the puncture caused by the needle heals.

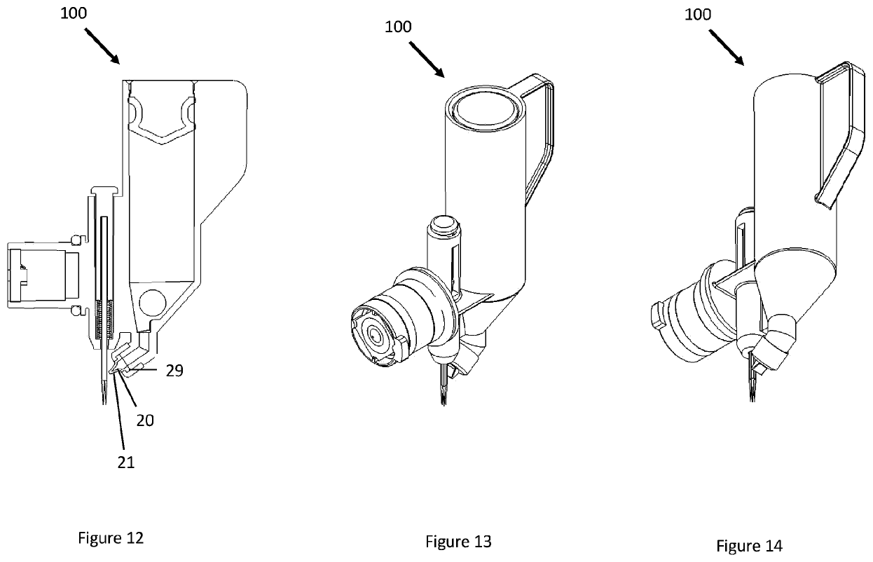

In the embodiments of FIGS. 1 and 2, the pigment nozzle 20 is dimensioned to be sealed by pigment surface tension, removing the need to have a valve, for example a duck bill valve. The surface tension sealing is aided by the viscosity of the pigment and the lack of air pressure within the reservoir. That is, the pigment is held within the nozzle by surface tension, without the need for any closure. FIGS. 12-14 shows various views of an alternative embodiment of a consumable cartridge 100, where the pigment nozzle 20 comprises a duck bill valve 21. The duck bill valve 21 is retained by valve retainer 29 in the form of a flange that the duck bill valve 21 resiliently deforms to pass over. The pigment nozzle 20 is during transmit capped with a cap that is removed before attachment of the consumable cartridge 100.

The consumable cartridge 10 comprises a mixing element 21 in the form of a mobile mixing element disposed in the pigment reservoir 18. The mobile mixing element 21, which may be a metal, glass or plastic pea, for example, is disposed in the pigment reservoir 18 for mixing the pigment. The animal marking apparatus may be configured to agitate the cartridge, causing the mixing element 21 to move within the pigment and mix it.

In the described embodiments but not necessarily all embodiments, there is an insufficient volume of the pigment in the pigment reservoir 18 to continue operating the consumable cartridge 10 after the needle 12 has blunted through use. The needle 12 will blunt after a certain number of penetrations thereby. The amount of ink required for a given number of penetrations for a particular needle can be determined, by experimenting with various volumes of pigment for example. Consequently, a user is required to replace a consumable cartridge 10 when the pigment is spent but the needle is not so blunt as to present an animal welfare issue.

Embodiments of the consumable cartridge 10 comprises non-volatile memory in the form of flash memory and electrical contacts in the form of electrical pads in electrical communication with the non-volatile memory for the animal marking machine to at least one of read and write to the non volatile memory. The non-volatile memory is mounted on a printed circuit board 43 housed in the cartridge joint member 40. The non-volatile memory holds at least one of:
Consumable cartridge type identification information, which can be used by the apparatus 11 to check that the right consumable cartridge is being used, and if not prevent operation of the cartridge;
consumable cartridge identification information, which can be associated with animal identification in a data store, so that which mice that were processed by the consumable cartridge 10 can be identified, and so cross contaminated mice can be identified;
information indicative of the number of pigment applications, which can be used to estimate or determine how many pigment applications by the consumable cartridge 10 remain so that appropriate action can be taken ahead of running out of ink;
information indicative of the number of symbols written, which can be used to estimate or determine how many more symbols may be written by the consumable cartridge 10.
pigment colour information, which can be used by the apparatus 11 to check that the right pigment colour is being used, and if not prevent operation of the cartridge;
needle type identification information, which can be used by the apparatus 11 to check the right type of needle is being used, and if not prevent operation of the cartridge;
date information, indicative of the manufacture date and/or last use date, for example;
total operation time information; and
generally any other useful information.

The consumable cartridge 10 comprises a 1-wire interface comprising electrical tracks 45 for the memory, comprising an electrical signal contact and an electrical ground.

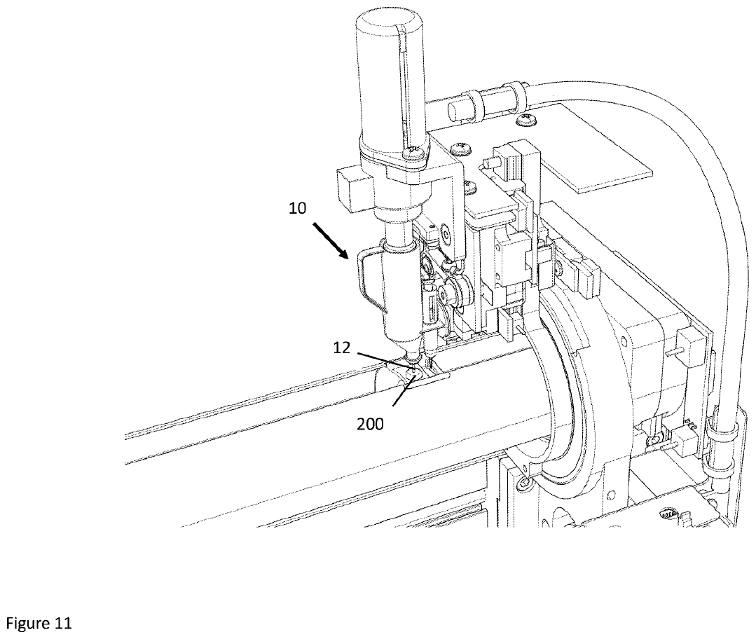

FIG. 11 shows a perspective view of the needle at a home dock or in a parked position. The needle is inserted into a pad 200 comprising polyurethane in the form of a Shore A 70 Dureometer cast polyurethane plug, however any penetrable material, for example silicon or another suitable polymer, may be used. When the needle in in the penetrating position, the needle is sealed, which may prevent the ink drying out and blocking the needle.

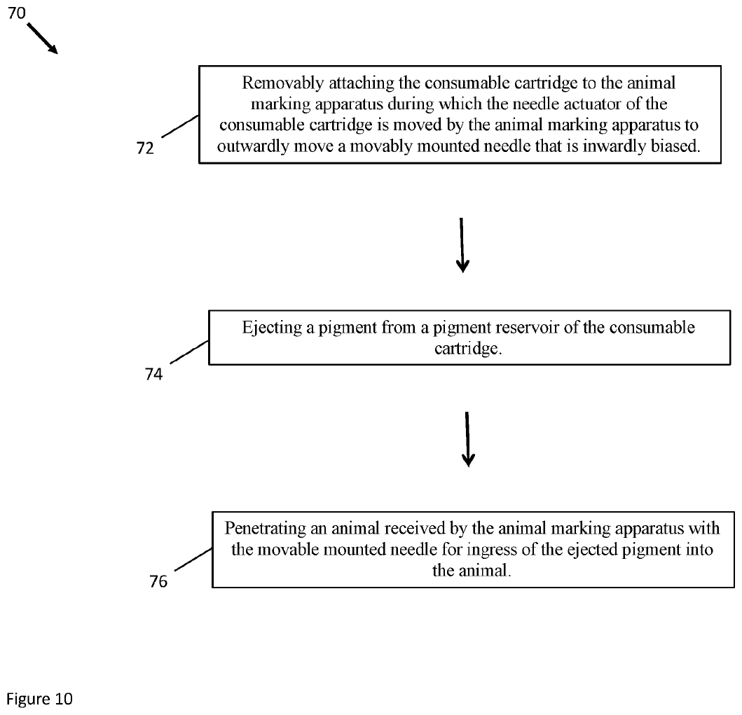

FIG. 10 shows a flow chart 70 for an embodiment of a method for marking an animal 114, comprising a plurality of steps. A step 72 comprises removably attaching the consumable cartridge 10 to the animal marking apparatus 11 during which the needle actuator 16 of the consumable cartridge 10 is moved by the animal marking apparatus 11 to outwardly move a movably mounted needle 12 that is inwardly biased. A step comprises ejecting a pigment from a pigment reservoir 18 of the consumable cartridge 10. A step comprises penetrating an animal 114 received by the animal marking apparatus 11 with the movable mounted needle 12 for ingress of the ejected pigment into the animal 114.

Now that embodiments have been described, it will be appreciated that some embodiments have some of the following advantages:
A user is required to replace a consumable cartridge when the pigment is spent but the needle is not so blunt as to present an animal welfare issue.
The needle is safely concealed until the cartridge is attached to the animal marking apparatus.
Information about the consumable cartridge and its use can be used to improved animal trial integrity, and a group of animals to which the same needle was applied can be identified, which may assist in tracing infections transferred by the same needle within the group.
The cartridge is not fiddly or time consuming to install.
The cartridge minimises pigment spills and smears.
The amount of pigment dispensed can be accurately controlled to minimise waste and cost.

Variations and/or modifications may be made to the embodiments described without departing from the spirit or ambit of the invention. The present embodiments are, therefore, to be considered in all respects as illustrative and not restrictive. Reference to a feature disclosed herein does not mean that all embodiments must include the feature.

Prior art, if any, described herein is not to be taken as an admission that the prior art forms part of the common general knowledge in any jurisdiction.

In the claims which follow and in the preceding description of the invention, except where the context requires otherwise due to express language or necessary implication, the word “comprise” or variations such as “comprises” or “comprising” is used in an inclusive sense, that is to specify the presence of the stated features but not to preclude the presence or addition of further features in various embodiments of the invention.

1. A consumable cartridge removably attachable to an animal marking apparatus, the consumable cartridge comprising:
a movably mounted needle and a biasing element operatively coupled to the movably mounted needle to withdraw the needle;
a needle actuator arranged to be moved by the animal marking apparatus during attachment of the cartridge and when so moved the needle actuator moves the movably mounted needle outwards; and
a pigment reservoir comprising a tattoo pigment, the pigment reservoir being in fluid communication with a pigment nozzle.

2. A consumable cartridge defined by claim 1 comprising a needle housing defining a cavity in which the movable mounted needle is withdrawn into and extending between an open end from which the needle actuator protrudes and another open end for passage of the needle, the needle actuator being arranged to be inwardly moved by the animal marking apparatus and so move the tip of the needle out of the other open end.

3. A consumable cartridge defined claim 2 wherein the biasing member comprises a compression spring that is coiled around the needle and having an end seated adjacent the other open end.

4. A consumable cartridge defined by claim 3 wherein the spring comprises another end that outwardly pushes the needle actuator.

5. A consumable cartridge defined by claim 4 wherein the needle actuator comprises an end cap attached to the movably mounted needle and having a seat for the other end of the spring.

6. A consumable cartridge defined by claim 2 comprising a cartridge joint member configured to be rotated into engagement with an animal marking apparatus joint member to form a connecting joint between the consumable cartridge and the animal marking apparatus, the needle actuator being arranged to be inwardly moved by a cam surface of the animal marking apparatus when the cartridge joint member is so rotated into engagement.

7. A consumable cartridge defined by claim 6 wherein the connecting joint comprises at least one of a connecting bayonet joint, and a connecting screw joint.

8. A consumable defined by claim 1 wherein the pigment reservoir comprises a plunger for moving the pigment.

9. A consumable defined by claim 7 wherein the plunger is externally actuatable by the animal marking apparatus.

10. A consumable cartridge defined by claim 1 wherein the pigment nozzle is adjacent to a tip of the needle.

11. A consumable cartridge defined by claim 10 wherein the pigment nozzle is directed at at least one of the movably mounted needle subsequent to being outwardly moved, and when removably attached to the animal marking apparatus, a point within the animal marking apparatus at which the animal present.

12. A consumable cartridge defined by claim 1 wherein the pigment nozzle comprises at least one of a duck-bill valve and a hollow needle.

13. A consumable cartridge defined by claim 1 wherein the pigment nozzle is dimensioned to be sealed by pigment surface tension.

14. A consumable cartridge defined by claim 1 comprising a mixing element disposed in the pigment reservoir.

15. A consumable cartridge defined by claim 14 comprising a mobile mixing element disposed in the pigment reservoir.

16. A consumable cartridge defined by claim 14 comprising non-volatile memory and electrical contacts in electrical communication with the non-volatile memory for the animal marking machine to at least one of read and write to the non volatile memory.

17. A consumable cartridge defined by claim 16 wherein the non-volatile memory comprises at least one of consumable cartridge identification information, information indicative of the number of pigment applications, pigment colour information, needle type identification information, date information, number of symbols marked on the animal, and total operation time information.

18. A consumable cartridge defined by claim 1 wherein the pigment reservoir holds no ejectable pigment when the needle is blunted through use.

19. A method for marking an animal, the method comprising the steps of:
removably attaching a consumable cartridge to an animal marking apparatus during which a needle actuator of the consumable cartridge is moved by the animal marking apparatus to outwardly move a movably mounted needle that is inwardly biased;
ejecting a pigment from a pigment reservoir of the consumable cartridge;
and penetrating an animal received by the animal marking apparatus with the movable mounted needle for ingress of the ejected pigment into the animal.

20. A consumable cartridge or method defined by claim 1 wherein the animal is a non-human animal.

21. (canceled)
